(19) United States
(12) Patent Application Publication
(10) Pub. No.: US 2009/0270841 A1
(43) Pub. Date: Oct. 29, 2009

(54) Catheters

(75) Inventor: David Christian Lentz, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/150,050

(22) Filed: Apr. 24, 2008

(51) Int. Cl. A61M 25/18; A61M 25/00

(52) U.S. Cl. 604/533; 604/523

(57) ABSTRACT

A catheter arrangement comprises a central tube with a wall of porous material, a proximal portion of which is covered with a non-porous layer. A hub member at the proximal end of the catheter arrangement has a port in fluid communication via the lumen of the central tube with the region surrounding the exposed distal end of the tube.

### FIELD OF INVENTION

The present invention relates to catheters for use in various medical procedures and in particular to catheters having walls through which fluids can pass.

### BACKGROUND OF THE INVENTION

One use of a catheter of the type described above is in an angiographic procedure in which a dye or other substance opaque to x-rays is injected into a body vessel or other site of medical interest. Existing catheters for delivering such fluids comprise a tubular member having walls with a plurality of mutually-spaced holes therethrough. The size and number of the holes together with the pressure of injection determine the flow rate of the fluid into the body.

A problem with existing catheters is that the creation of a hole in a catheter wall also creates a weakened zone around the location of the hole. This means that one cannot have holes all the way round a catheter and that the total number of holes created is limited if the catheter is to remain self-supporting.

### SUMMARY OF INVENTION

Aspects of the present invention seek to overcome or reduce the above problem.

According to a first aspect of the present invention there is provided a catheter comprising a tubular member with a longitudinal extent and having a wall of a porous nature, a part of the longitudinal extent of the tubular member being covered with a layer of non-porous material.

According to a second aspect of the present invention there is provided a catheter comprising a tubular member with a longitudinal extent and having a wall which is porous along its entire length and around its entire periphery, a part of the longitudinal extent of the porous tubular member being covered with a layer of non-porous material.

According to a third aspect of the present invention there is provided a catheter arrangement comprising a tubular member with a longitudinal extent and having a wall comprising a plurality of wires wound so as to permit fluid flow therebetween, the tubular member having a central lumen and a proximal part of the longitudinal extent of the tubular member being covered with a layer of non-porous material, the catheter arrangement further comprising a hub member connected to a proximal end of the tubular member, said hub member having a port in fluid communication with said central lumen.

In preferred embodiments the wall of the tubular member is of wound, woven or braided wire, which may be of stainless steel.

The non-porous material is preferably a heat-shrinkable material such as poly ether block amide.

### BRIEF DESCRIPTION OF THE DRAWINGS

Preferred embodiments of the present invention will now be described, by way of example only, with reference to the accompanying drawings of which:

FIG. 1 is a side view of a catheter arrangement in accordance with a first embodiment of the present invention;

FIG. 2 is an enlarged view of part of a catheter tube of the arrangement of FIG. 1;

FIG. 3 is a cross-sectional view of the catheter arrangement of FIG. 1 in combination with a guide wire arrangement; and

FIG. 4 is a cross-sectional view of the catheter arrangement of FIG. 1 in combination with an alternative guide wire arrangement.

### DESCRIPTION OF THE PREFERRED EMBODIMENTS

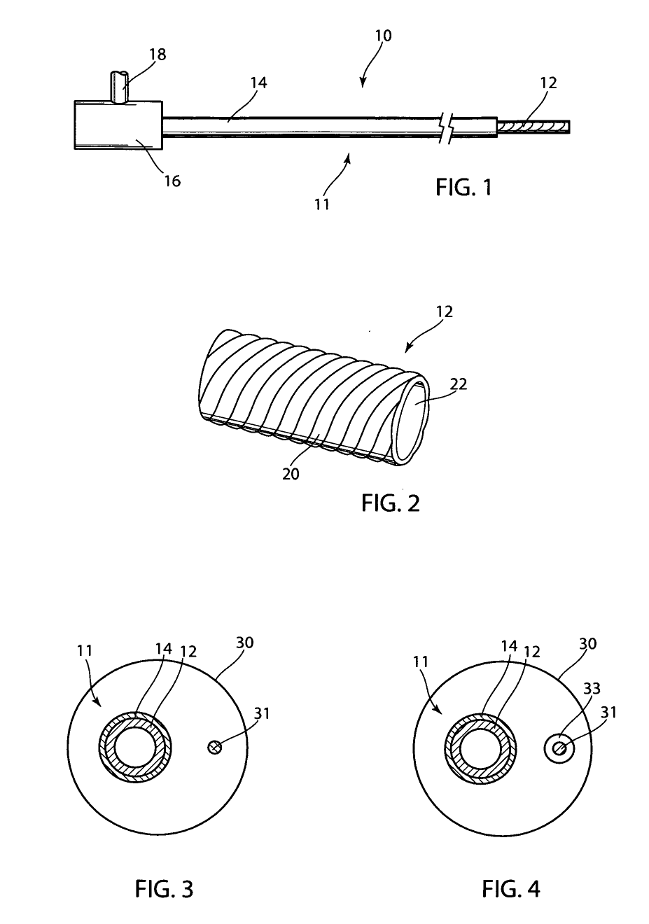

Referring to the drawings, FIG. 1 shows an angiographic catheter arrangement 10 comprising a catheter 11. The catheter 11 comprises a central catheter tube 12, a proximal part of which is surrounded by a layer 14 of non-porous tubing. The central tube 12 extends for the entire length of the catheter 11 and is attached at its proximal end to a hub 16. Hub 16 comprises inlet means, shown schematically at 18, for the introduction of dyes or other fluids to the catheter tube 12.

The catheter tube 12 is in the form of a hollow wire rope (or cable tube) comprising stainless steel wires 20 wound around a central lumen 22. Such a cable tube is available from Asahi Intecc Co. Ltd under the name ACTONE (Trade Mark). The end 15 of the tube is closed by sealing it. The tube 12 has an exterior diameter of between 0.3 mm and 1.3 mm. The wall of the tube comprises outwardly from the central lumen between 6 and 18 wires extending along its length. It will be appreciated that, in use, fluid passes between the individual wires forming the wall. A preferred material is stainless steel type 304.

The layer of tubing 14 is preferably heat-shrunk around the tube 12. A preferred material is poly ether block amide. The tubing 14 is arranged to prevent flow of fluid through the wall of tube 12 except where tube 12 is not covered by tubing 14.

FIG. 3 shows a cross-section view of the arrangement of FIG. 1 in combination with a guide wire 31 within a common lumen 30. As is well-known, radio-opaque marker elements (not shown) may be applied to the various components to enable their position within the patient to be monitored.

In use, guide wire 31 is introduced to a site of medical interest and lumen 30 catheter arrangement 10 therein is passed along the guide wire until the exposed portion of catheter tube 12 reaches the desired position of dye release. Dye is then supplied to inlet 18 from which it passes along the interior of tube 12 until it reaches the exposed distal end portion of tube 12. From here it passes through the porous walls of tube 12 to the location of angiographic interest.

An advantage of the above-described embodiment is that the wall of the tube 12 is free from weak points. Because the tube is self-supporting, the desired degrees of flexibility and porosity may be obtained without weakening the wall structure. In addition, since dye passes through passages along the entire length of, and around the entire circumference of, the exposed surface of tube 12, a uniform distribution of dye is readily obtained. Tube 12 also provides good flexibility. It also has good torque characteristics, kink resistance, compression resistance and shape recovery characteristics.

Various modifications may be made to the above-described embodiment.

For example, the distal end of tube 12 may be partly or wholly open if desired; this would serve to increase the rate of fluid flow, but with some loss of uniformity in the flow.

Instead of type 304, stainless steel type 316 can be used for the wire forming tube 12.

The distal tip of tube 12 may have a pig-tail or other curved shape to assist in insertion of the catheter arrangement and to maintain the correct position thereof.

The tube 12 does not need to extend along the entire length of catheter 11. Tube 12 may be connected to hub 16 by means of an intermediate tube.

The radio-opaque marker elements can be omitted.

The walls of the tube may be of braided or woven wire.

Other metallic or non-metallic materials may be used for the wires of the walls of tube 12. In other modifications, instead of wires 20, the walls of the tube are made of a material which is itself porous, e.g. a foamed plastic material or a porous ceramic material. Because of its porous nature, the material allows fluid to flow out over its entire exposed length.

The tubing 14 may be of a different non-porous polymer or, indeed, any suitable non-porous material.

If desired one or more additional lengths of tubing 14 may be applied to the tube 12 so that dye can emerge from two or more portions of the exterior of tube 12.

The guide wire 31 may extend along the entire length of lumen 30; alternatively lumen 30 may surround only a distal portion of the guide wire, with the guide wire emerging from the lumen 30 at an intermediate point thereof.

FIG. 4 shows a modification in which guide wire 31 has its own guide wire lumen 33.

In addition to angiography the above-described embodiments can also be employed in thrombectomy, in which case, at appropriate parts of the procedure, one or more anti-clotting agents can be delivered by the catheter arrangement to a desired site. Alternatively, the catheter arrangement can be used in an aspiration procedure. In this case inlet 18 becomes an outlet and suction applied thereto enables the exposed distal portion of tube 11 to be employed as an aspirator to remove fluids from its surroundings.

The catheter arrangement can be used to supply fluid and as an aspirator at successive stages of a single operation; it is merely necessary to connect appropriate tubing to inlet/outlet 18. This has the advantage of reducing trauma for a patient by the removal and insertion of additional catheters.

It will be understood that the above description of the present invention is susceptible to various modification, changes and adaptations.

What is claimed is:

1. A catheter comprising a tubular member with a longitudinal extent and having a wall of a porous nature, a part of the longitudinal extent of the tubular member being covered with a layer of non-porous material.

2. A catheter according to claim 1, wherein the wall of the tubular member comprises a plurality of wires.

3. A catheter according to claim 2, wherein the wires are selected from:
wound wires, woven wires, or braided wires.

4. A catheter according to claim 2, wherein the wires are made of stainless steel.

5. A catheter according to claim 1, wherein the non-porous material is of heat shrinkable material.

6. A catheter according to claim 5, wherein the non-porous material is poly ether block amide.

7. A catheter according to claim 1, wherein the wall of the tubular member is of a porous material.

8. A catheter comprising a tubular member with a longitudinal extent and having a wall which is porous along its entire length and around its entire periphery, a part of the longitudinal extent of the porous tubular member being covered with a layer of non-porous material.

9. A catheter arrangement according to claim 1, wherein the tubular member has a central lumen and wherein the non-porous material covers a proximal part of the longitudinal extent of the tubular member, and the catheter arrangement further comprises a hub member connected to a proximal end of the tubular member, said hub member having a port in fluid communication with said central lumen.

10. A catheter arrangement comprising a tubular member with a longitudinal extent and having a wall comprising a plurality of stainless steel wires wound so as to permit fluid flow therebetween, the tubular member having a central lumen and a proximal part of the longitudinal extent of the tubular member being covered with a layer of non-porous heat shrinkable material, the catheter arrangement further comprising a hub member connected to a proximal end of the tubular member, said hub member having a port in fluid communication with said central lumen.
